(19) United States
(12) Patent Application Publication
(10) Pub. No.: US 2012/0115141 A1
(43) Pub. Date: May 10, 2012

(54) ENDPOINT TAQMAN METHODS FOR DETERMINING ZYGOSITY OF COTTON COMPRISING Cry1F EVENT 281-24-236

(75) Inventors: Wei Chen, Carmel, IN (US); Wesley A. Marchione, Greenfield, IN (US)

(73) Assignee: Dow AgroSciences LLC, Indianapolis, IN (US)

(21) Appl. No.: 13/267,644

(22) Filed: Oct. 6, 2011

Related U.S. Application Data
(60) Provisional application No. 61/390,858, filed on Oct. 7, 2010.

(51) Int. Cl. C12Q 1/68

(52) U.S. Cl. 435/6.11

(57) ABSTRACT

A method for zygosity analysis of the cotton Cry1F event 281-24-236 is provided. The method provides 281-24-236 event-specific and cotton endogenous reference gene-specific primers and TaqMan probe combinations for use in an endpoint biplex TaqMan PCR assay capable of determining event zygosity and for assisting in event introgression and breeding.

### CROSS REFERENCE TO RELATED APPLICATIONS

This application claims priority to U.S. Provisional Patent Application Ser. No. 61/390,858, filed Oct. 7, 2010, the entire disclosure of which is expressly incorporated herein by reference.

### BACKGROUND OF THE INVENTION

WIDESTRIKE is a commercial cotton product comprising two synthetic Bacillus thuringiensis gene events: a cry1Ac-based event 3006-210-23 and a cry1F-based event 281-24-236, which together provide broad spectrum resistance to insect attack. The events are discussed in more detail in, for example, U.S. Pat. No. 7,179,965.

Cotton is an allotetraploid species which contains one A-subgenome and one D-subgenome per haploid chromosome. Transgenic WIDESTRIKE cotton plants contain a single copy event of the transgene insert in only one of the two subgenomes. Since the two subgenomes have high similarity in nucleotide sequences, oligonucleotide primers and probes specific for the null allele (designed from the flanking region of the transgene inserted into one subgenome) often amplify the fragment in the other subgenome. As such, it can be difficult to differentiate wild-type samples from plant samples that contain the transgene. Nevertheless, these technical difficulties were overcome, and the development and validation of reagents and conditions that can be used for a TaqMan zygosity assay of WIDESTRIKE Event 281-24-236 are described below.

Various methods can be used to detect the presence of a given event in a sample. One example is the Pyrosequencing technique as described by Winge (Innov. Pharma. Tech. 00:18-24, 2000). In this method an oligonucleotide is designed that overlaps the adjacent genomic DNA and insert DNA junction. The oligonucleotide is hybridized to single-stranded PCR product from the region of interest (one primer in the inserted sequence and one in the flanking genomic sequence) and incubated in the presence of a DNA polymerase, ATP, sulfurylase, luciferase, apyrase, adenosine 5′ phosphosulfate and luciferin. DNTPs are added individually and the incorporation results in a light signal that is measured. A light signal indicates the presence of the transgene insert/flanking sequence due to successful amplification, hybridization, and single or multi-base extension. (This technique is usually used for initial sequencing, not for detection of a specific gene when it is known.)

Fluorescence Polarization is another method that can be used to detect an amplicon. Following this method, an oligonucleotide is designed to overlap the genomic flanking and inserted DNA junction. The oligonucleotide is hybridized to single-stranded PCR product from the region of interest (one primer in the inserted DNA and one in the flanking genomic DNA sequence) and incubated in the presence of a DNA polymerase and a fluorescent-labeled ddNTP. Single base extension results in incorporation of the ddNTP. Incorporation can be measured as a change in polarization using a fluorometer. A change in polarization indicates the presence of the transgene insert/flanking sequence due to successful amplification, hybridization, and single base extension.

The Invader assay (Third Wave Technologies, now Hologic, Inc., WI, USA) is a non-PCR based method and involves denaturing genomic DNA (25-50 min), preparing the Invader assay plates (adding mix, controls, standards, and DNA), incubating the plates on Thermo Cyclers or incubators (2-2½ hours), and reading the assay plate on the Tecan plate reader. The Invader assay, although novel in its kinetics, has many limitations. It is very time consuming and labor-intensive. Since it is a non-PCR based assay, it requires high-quality DNA and the result is highly variable if the concentration of DNA is sub-optimal (<11 ng/μl). If insufficient separation of RFU (relative fluorescence units) values is observed, an additional 30 minute incubation period is required and the plate will then be re-read.

TAQMAN (Roche Molecular Systems; see also e.g., Life Technologies Corp., Carlsbad, Calif.) is a method of detecting and quantifying the presence of a DNA sequence. Briefly, a FRET oligonucleotide probe is designed with one oligo within the transgene and one in the flanking genomic sequence for event-specific detection. The FRET probe and PCR primers (one primer in the insert DNA sequence and one in the flanking genomic sequence) are cycled in the presence of a thermostable polymerase and dNTPs. Hybridization of the FRET probe results in cleavage and release of the fluorescent moiety away from the quenching moiety on the FRET probe. A fluorescent signal indicates the presence of the flanking/transgene insert sequence due to successful amplification and hybridization.

Molecular Beacons have been described for use in sequence detection. Briefly, a FRET oligonucleotide probe is designed that overlaps the flanking genomic and insert DNA junction. The unique structure of the FRET probe results in it containing secondary structure that keeps the fluorescent and quenching moieties in close proximity. The FRET probe and PCR primers (one primer in the insert DNA sequence and one in the flanking genomic sequence) are cycled in the presence of a thermostable polymerase and dNTPs. Following successful PCR amplification, hybridization of the FRET probe to the target sequence results in the removal of the probe secondary structure and spatial separation of the fluorescent and quenching moieties. A fluorescent signal indicates the presence of the flanking genomic/transgene insert sequence due to successful amplification and hybridization.

Another challenge, among many, is finding a suitable reference gene for a given test. For example, as stated in the abstract of Czechowski et al., “An exceptionally large set of data from Affymetrix ATH1 whole-genome GeneChip studies provided the means to identify a new generation of reference genes with very stable expression levels in the model plant species Arabidopsis (Arabidopsis thaliana). Hundreds of Arabidopsis genes were found that outperform traditional reference genes in terms of expression stability throughout development and under a range of environmental conditions.” (Czechowski et al. (2005) Genome-wide identification and testing of superior reference genes for transcript normalization in Arabidopsis. Plant Physiol. 139, 5-17.)

Brodmann et al. (2002) relates to real-time quantitative PCR detection of transgenic maize content in food for four different maize varieties approved in the European Union. Brodmann, P. D., P. D., Ilg E. C., Berthoud H., and Herrmann, A. Real-Time Quantitative Polymerase Chain Reaction Methods for Four Genetically Modified Maize Varieties and Maize DNA Content in Food. J. of AOAC international 2002 85 (3).

Baeumler et al. relates to a real-time quantitative PCR detection method specific to WIDESTRIKE transgenic cotton. J. Agric. Food Chem. (2006) 54(18), 6527-6534.

Hernandez et al. (2004) mentions four possible genes for use with real-time PCR. Hernandez, M., Duplan, M.-N., Berthier, G., Vaitilingom, M., Hauser, W., Freyer, R., Pla, M., and Bertheau, Y. Development and comparison of four real-time polymerase chain reaction systems for specific detection and quantification of Zea mays L. J. Agric. Food Chem. 2004, 52, 4632-4637.

Costa et al. (2007) looked at these four genes (also in the real-time PCR context) and concluded that the alcohol dehydrogenase and zein genes were the best reference genes for detecting a sample “event” (a lectin gene) for transgenic feed intermix issues. Costa, L. D., and Martinelli L. Development of a Real-Time PCR Method Based on Duplo Target Plasmids for Determining an Unexpected Genetically Modified Soybean Intermix with Feed Components. J. Agric. Food Chem. 2007, 55, 1264-1273.

Huang et al. (2004) used plasmid pMulM2 as reference molecules for detection of MON810 and NK603 transgenes in maize. Huang and Pan, “Detection of Genetically Modified Cotton MON810 and NK603 by Multiplex and Real-Time Polymerase Chain Reaction Methods,” J. Agric. Food Chem., 2004, 52 (11), pp 3264-3268.

Gasparic et al. (2008) suggest LNA technology, from a comparison to cycling probe technology, TaqMan, and various real-time PCR chemistries, for quantitatively analyzing cotton events (such as MON810). Ga\check{s}pari\check{e}, Cankar, \check{Z}el, and Gruden, “Comparison of different real-time PCR chemistries and their suitability for detection and quantification of genetically modified organisms,” BMC Biotechnol. 2008; 8: 26.

US 20070148646 relates to a primer extension method for quantification that requires controlled dispensation of individual nucleotides that can be detected and quantified by the amount of nucleotides incorporated. This is different from the TaqMan PCR method using an internal reference gene.

To distinguish between homozygous and hemizygous genotypes of corn event TC1507, an Invader assay has been successfully used for this event. Gupta, M., Nirunsuksiri, W., Schulenberg, G., Hartl, T., Novak, S., Bryan, J., Vanopdorp, N., Bing, J. and Thompson, S. A non-PCR-based Invader Assay Quantitatively Detects Single-Copy Genes in Complex Plant Genomes. Mol. Breeding 2008, 21, 173-181.

Huabang (2009) relates to PCR-based zygosity testing of transgenic maize. However, no reference gene appears to be used. Huabang, “An Accurate and Rapid PCR-Based Zygosity Testing Method for Genetically Modified Maize,” Molecular Plant Breeding, 2009, Vol. 7, No. 3, 619-623.

### BRIEF SUMMARY OF THE INVENTION

The present invention relates in part to a molecular assay for determining zygosity of event 281-24-236 in cotton. More specifically, the present invention relates in part to an endpoint TaqMan PCR assay for a WIDESTRIKE cry1F event 281-24-236 in cotton utilizing endogenous reference genomic DNA in cotton. Some embodiments are directed to assays that are capable of high throughput zygosity analysis. The subject assays offer a reliable, consistent, and cost effective option. Some preferred assays are fluorescence-based and consist of a PCR set-up with the plate being subsequently read on a plate reader. This eliminates the denaturation step and the need for long incubation periods. Thus, the subject assays improve time, labor and cost efficiency.

The subject assays can be used for event-specific (281-24-236) detection of the cry1F transgene in cotton plants by end-point TaqMan PCR. This TaqMan PCR based zygosity assay is a biplex assay. Some of the subject assays use oligonucleotides specific to the 281-24-236 event and its flanking genomic sequences; some other assays utilize oligonucleotides corresponding to cotton genomic DNA used as a reference sequence in a single reaction. Zygosity is determined by the relative intensity of fluorescence specific for Event 281-24-236 to the reference sequence.

This 281-24-236 event-specific assay amplifies a 147-bp fragment, unique to the event resulting from the insertion of the 281-24-236 construct cassette into the cotton genomic DNA. A target-specific oligonucleotide probe (28_probe) binds to the target between two event-specific 281-24-236 PCR primers (WT_F1, complementary to the flanking genomic sequences; 281_R1, complementary to the 5′ end of the 281-24-236 insertion) and is labeled with fluorescent dye: FAM as a reporter dye at its 5′ end and MGBNFQ (minor grove binding non-fluorescent quencher) as a quencher at its 3′ end. PCR products are measured after optimal number of cycles, when the reaction is in the early exponential phase.

Due to the allotetraploid nature of the cotton and the molecular structure of the Event 281-24-236, traditionally designed oligos specific for the flanking cotton genomic sequences of this event would amplify in all individual plants of segregating populations, and thus could not be used to distinguish wild-type plants from transgenic plants in a plus/minus matter. However, the oligos disclosed below can be used to identify wild-type plants from transgenic plants in a plus/minus matter. Moreover, the zygosity of the transgenic plants can also be determined using these oligos. The genome sequence specific oligos were used as a reference for relative quantitation of genomic DNA. This flanking region specific assay amplifies a 186-bp fragment of the genomic sequences with a pair of oligos (WT_F1 and WT_R2) and a probe (WT_probe) labeled with VIC and MGBNFQ.

This is a fluorescence-based end-point assay that allows the results to be directly read in a plate reader for identification of Event 281-24-236 in cotton. The amplicons generated are not intended to be resolved on agarose gels.

In some preferred embodiments, the subject protocols are preferably used for breeding applications, i.e., testing related to introgression of a WIDESTRIKE event into other cotton lines.

### BRIEF DESCRIPTION OF THE DRAWINGS

FIG. 1 shows components of the cry1F event 281-24-236 insert and its border sequences.

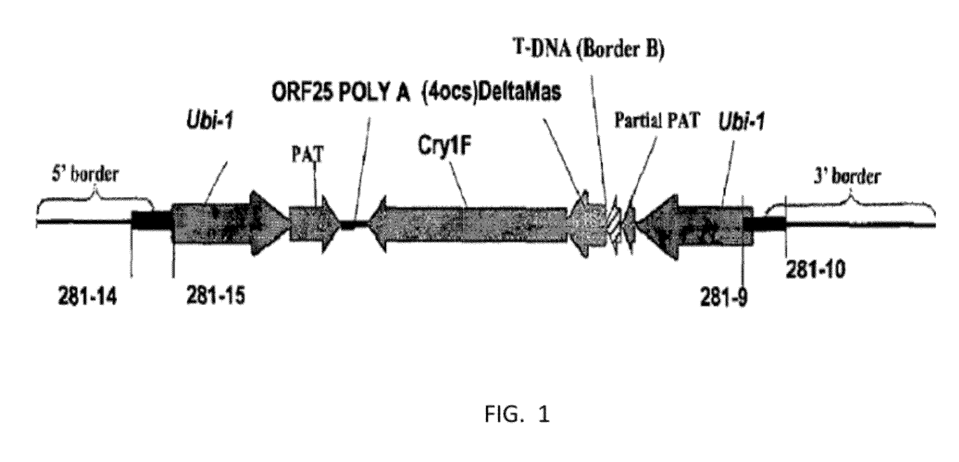

FIG. 2 illustrates location of the primer and probe sequences with respect to the subgenomes and trangene insert.

FIG. 3 shows an example of a distribution graph between sample numbers and absolute ratios of SOB1/SOB2.

### BRIEF DESCRIPTION OF THE SEQUENCES

SEQ ID NO:1 is the DNA sequence for the cry1F event 281-24-236 insert and its border sequences.

SEQ ID NOs:2-6 are exemplified primers and probes for use according to the subject invention.

SEQ ID NO:7 is the transgene amplicon.

SEQ ID NO:8 is the wild-type amplicon.

### DETAILED DESCRIPTION OF THE INVENTION

Cotton Events 281-24-236 (Cry1F) and 3006-210-23 (Cry1Ac), are two independent transgenic events of WIDESTRIKE trait (trademark of Dow AgroSciences LLC). WIDESTRIKE varieties express both Cry1F and Cry1Ac insecticidal proteins and provide excellent season-long protection against a broad spectrum of insect pests (Baeumler et al. 2006). To characterize these two events for transgene dosage, i.e., determination of “zygosity” (distinguishing homozygous genotype from hemizygous and null genotypes), transgene analysis previously relied on Invader assays. Invader assays have been used successfully in many transgenic plants. However, it is very sensitive to DNA quality and quantity, and it does not always yield robust enough separation for zygosity analysis if the DNAs are not handled properly, thus leading to significant investment in reruns. Moreover, there was a tremendous increase in the volume of samples to be analyzed.

Application of the TaqMan assay was complicated since cotton is an allotetraploid species containing two subgenomes, A and D, in its nucleus. Transgenic cotton plants chosen for further characterization contain a single copy event with the transgene insert present in only one of the two subgenomes. The two subgenomes have very high similarity in nucleotide sequences. Oligos based on the junction region between the insert and cotton genome for detecting wild type alleles often amplify the fragment in both subgenomes. Since transgene samples contain one intact subgenome, very often with fragments matching to the oliogos from the junction thus can hybridize to the oligos and make it difficult to differentiate wild type samples from transgene homozygotes. This is no exception for Event 281-24-236. Oligos specific to the flanking cotton genomic sequences amplified in all individual plants, whether they are homozygous transgenic or not. Thus it cannot be used to separate wild-type allele from transgene allele directly. However, homozygous, hemizygous and wild type samples have unique copy numbers of the amplicon from the cotton genome specific oligos. It can be used as copy number control for endpoint TaqMan assay design to differentiate transgenic samples from wild type.

Thus, the subject invention relates in part to a fluorescence-based endpoint TaqMan PCR assay utilizing endogenous reference cotton genomic DNA as a (copy number) control for high-throughput zygosity analysis of 281-24-236, a cotton Cry1F event.

The subject invention also relates in part to the development of a biplex endpoint TaqMan PCR for 281-24-236 event specific zygosity analysis. Further, the subject invention relates in part to the development of 281-24-236 breeding test kits.

Endpoint TaqMan assays are based on a plus/minus strategy, by which a “plus” signifies the sample is positive for the assayed gene and a “minus” signifies the sample is negative for the assayed gene. These assays typically utilize two sets of oligonucleotides for identifying the 281-24-236 transgene sequence and the wild-type gene sequence respectively, as well as dual-labeled probes to measure the content of transgene and wild type sequence.

Although the Invader assay has been a robust technique for characterizing events, it is very sensitive to DNA quality. In addition, the assay requires a high quantity of DNA. Invader also requires an additional denaturing step which, if not handled properly, can render the Invader assay unsuccessful. Additionally, the longer assay time of the Invader assay is limited in its flexibility to efficiently handle large numbers of 281-24-236 samples for analysis in a commercial setting. One main advantage of the subject invention is time savings and elimination of the denaturing step. The subject Endpoint TaqMan analysis for detecting 281-24-236 events offers surprising advantages over Invader, particularly in analyzing large number of samples.

In one embodiment, the 281-24-236 event-specific PCR reaction amplifies a 147-bp fragment, unique to the event, resulting from the insertion of the 281-24-236 construct cassette into the cotton genomic DNA. A 281-24-236 target-specific oligonucleotide probe binds to the target between two event-specific PCR primers and is labeled with a fluorescent dye and quencher. Possible fluorescent labels include FAM as a reporter dye at the 281-24-236 probe 5′ end and a Black Hole Quencher 1 (BHQ1) as the quencher at the 281-24-236 probe 3′ end.

Using a range of empirical factors and judgment, a reference reaction was designed for the same location of the genome, without the presence of the transgene. This reaction was selected as reference DNA for the 281-24-236 zygosity analysis. Gene specific primers and a gene-specific probe, in one embodiment labeled with VIC at the probe 5′ end and a Black Hole Quencher 2 (BHQ2) at the probe 3′ end, were assessed for use in rapid quantification for the cotton endogenous genes assessed as possible reference genes for the 281-24-236 zygosity analysis.

The gene-specific primers and probes for the Cry1F gene and the cotton endogenous DNA were tested for PCR efficiencies. Primer and probe combinations for the cotton endogenous DNA were exploited for multiplexing capabilities and endpoint TaqMan zygosity assay.

In some embodiments, the subject zygosity assays utilize a biplex of oligonucleotides specific to the 281-24-236 event and to the cotton endogenous DNA lacking the event (the flanking sequences lacking the subject cry1F insertion) in the same amplification assay. Zygosity is determined by the relative intensity of fluorescence specific for Event 281-24-236 as compared to the reference DNA.

In some embodiments, the 281-24-236 event-specific assay amplifies a 147-bp fragment, unique to the event, resulting from the insertion of the 281-24-236 construct cassette into the cotton genomic DNA. A target-specific oligonucleotide probe binds to the target between two event-specific 281-24-236 PCR primers and is labeled with two fluorescent dyes: FAM as a reporter dye at its 5′ end and BHQ as a quencher dye at its 3′ end. PCR products are measured after optimal number of cycles, typically when the reaction is in the early exponential phase.

In some embodiments, the cotton-specific reference system amplifies a 186 bp fragment for the reference reaction. A pair of wild type specific oligos and a wild type specific probe labeled with VIC at the 3′ end and a BHQ at the 5′ end are used for rapid quantitation.

In some embodiments, the fluorescence-based end-point TaqMan assay for 281-24-236 zygosity analysis allows the results to be directly read in a plate reader for identification of the WIDESTRIKE Event 281-24-236 in cotton and the reference gene.

The subject invention includes breeding applications such as testing the introgression of WIDESTRIKE into other cotton lines.

Detection methods and kits of the subject invention can be used to identify events according to the subject invention. Methods and kits of the subject invention can be used for accelerated breeding strategies and to establish linkage data.

Detection techniques of the subject invention are especially useful in conjunction with plant breeding, to determine which progeny plants comprise a given event, after a parent plant comprising an event of interest is crossed with another plant line in an effort to impart one or more additional traits of interest in the progeny. These TaqMan PCR analysis methods benefit cotton breeding programs as well as quality control, especially for commercialized transgenic cotton seeds. TaqMan PCR detection kits for these transgenic cotton lines can also now be made and used. This can also benefit product registration and product stewardship.

Still further, the subject invention can be used to study and characterize transgene integration processes, genomic integration site characteristics, event sorting, stability of transgenes and their flanking sequences, and gene expression (especially related to gene silencing, transgene methylation patterns, position effects, and potential expression-related elements such as MARS [matrix attachment regions], and the like).

This invention further includes processes of making crosses using a 281-24-236 plant as at least one parent. For example, the subject invention includes an F₁ hybrid plant having as one or both parents any of the plants exemplified herein. This invention includes a method for producing an F₁ hybrid seed by crossing an exemplified plant with a different (e.g. in-bred parent) plant, harvesting the resultant hybrid seed, and testing the seed/plant sample according to the subject invention. Characteristics of the resulting plants may be improved by careful consideration of the parent plants.

An insect-resistant cotton plant can be bred by first sexually crossing a first parental cotton plant consisting of a cotton plant grown from seed of any one of the lines referred to herein, and a second parental cotton plant, thereby producing a plurality of first progeny plants; and then selecting a first progeny plant that is resistant to insects (or that possesses at least one of the events of the subject invention); and selfing the first progeny plant, thereby producing a plurality of second progeny plants; and then selecting from the second progeny plants a plant that is resistant to insects (or that possesses at least one of the events of the subject invention). These steps can further include the back-crossing of the first progeny plant or the second progeny plant to the second parental cotton plant or a third parental cotton plant. A cotton crop comprising cotton seeds of the subject invention, or progeny thereof, can then be planted.

It is also to be understood that two different transgenic plants can also be mated to produce offspring that contain two independently segregating added, exogenous genes. Selfing of appropriate progeny can produce plants that are homozygous for both added, exogenous genes. Back-crossing to a parental plant and out-crossing with a non-transgenic plant are also contemplated, as is vegetative propagation. Other breeding methods commonly used for different traits and crops are known in the art. Backcross breeding has been used to transfer genes for a simply inherited, highly heritable trait into a desirable homozygous cultivar or inbred line, which is the recurrent parent. The source of the trait to be transferred is called the donor parent. The resulting plant is expected to have the attributes of the recurrent parent (e.g., cultivar) and the desirable trait transferred from the donor parent. After the initial cross, individuals possessing the phenotype of the donor parent are selected and repeatedly crossed (backcrossed) to the recurrent parent. The resulting parent is expected to have the attributes of the recurrent parent (e.g., cultivar) and the desirable trait transferred from the donor parent.

DNA molecules of the present invention can be used with molecular markers in a marker assisted breeding (MAB) method. DNA molecules of the present invention can be used with methods (such as, AFLP markers, RFLP markers, RAPD markers, SNPs, and SSRs) that identify genetically linked agronomically useful traits, as is known in the art. The insect-resistance trait can be tracked in the progeny of a cross with a cotton plant of the subject invention (or progeny thereof and any other cotton cultivar or variety) using the MAB methods. The DNA molecules are markers for this trait, and MAB methods that are well-known in the art can be used to track the insect-resistance trait(s) in cotton plants where at least one cotton line of the subject invention, or progeny thereof, was a parent or ancestor. The methods of the present invention can be used to identify any cotton variety having the insect-resistance event from cotton line 281-24-236.

Methods of the subject invention include a method of producing an insect-resistant cotton plant wherein said method comprises breeding with a plant of the subject invention. More specifically, said methods can comprise crossing two plants of the subject invention, or one plant of the subject invention and any other plant, and tracking the subject event according to the subject invention. Preferred methods further comprise selecting progeny of said cross by analyzing said progeny for an event detectable according to the subject invention.

A preferred plant, or a seed, propagated and developed according to the subject invention comprises in its genome at least one of the insert sequences, as identified in Table 1, together with at least 20-500 or more contiguous flanking nucleotides on both sides of the insert, as identified in Table 1. Unless indicated otherwise, “event 281-24-236” or like reference refers to DNA of SEQ ID NO:1 that includes the heterologous DNA inserted in the genomic location identified by all or part of both of the flanking genomic sequences immediately adjacent to the inserted DNA that would be expected to be transferred to progeny that receives the inserted DNA as a result of a sexual cross of a parental line that includes the event.

Definitions and examples are provided herein to help describe the present invention and to guide those of ordinary skill in the art to practice the invention. Unless otherwise noted, terms are to be understood according to conventional usage by those of ordinary skill in the relevant art. The nomenclature for DNA bases as set forth at 37 CFR §1.822 is used.

A transgenic “event” is produced by transformation of plant cells with heterologous DNA, i.e., a nucleic acid construct that includes a transgene of interest, regeneration of a population of plants resulting from the insertion of the transgene into the genome of the plant, and selection of a particular plant characterized by insertion into a particular genome location. The term “event” refers to the original transformant and progeny of the transformant that include the heterologous DNA. The term “event” also refers to progeny produced by a sexual outcross between the transformant and another variety that includes the genomic/transgene DNA. Even after repeated back-crossing to a recurrent parent, the inserted transgene DNA and flanking genomic DNA (genomic/transgene DNA) from the transformed parent is present in the progeny of the cross at the same chromosomal location. The term “event” also refers to DNA from the original transformant and progeny thereof comprising the inserted DNA and flanking genomic sequence immediately adjacent to the inserted DNA that would be expected to be transferred to a progeny that receives inserted DNA including the transgene of interest as the result of a sexual cross of one parental line that includes the inserted DNA (e.g., the original transformant and progeny resulting from selfing) and a parental line that does not contain the inserted DNA.

A “junction sequence” spans the point at which DNA inserted into the genome is linked to DNA from the cotton native genome flanking the insertion point, the identification or detection of one or the other junction sequences in a plant's genetic material being sufficient to be diagnostic for the event. Included are the DNA sequences that span the insertions in herein-described cotton events and similar lengths of flanking DNA. Specific examples of such diagnostic sequences are provided herein; however, other sequences that overlap the junctions of the insertions, or the junctions of the insertions and the genomic sequence, are also diagnostic and could be used according to the subject invention.

Primers, amplicons, and probes can be designed for use according to the subject invention based in part on the flanking, junction, and/or insert sequences. Related primers and amplicons can be included as components of the invention. PCR analysis methods using amplicons that span across inserted DNA and its borders can be used to detect or identify commercialized transgenic cotton varieties or lines derived from the subject proprietary transgenic cotton lines.

The sequence of the cry1F insert (together with regulatory sequences), flanked by the flanking sequences is provided as SEQ ID NO:1. Table 1 provides the coordinates of the insert and flanking sequences with respect to SEQ ID NO:1.

TABLE 1

| | Residue location in SEQ ID NO: 1: | | |
|---|---|---|---|
| Event | 5′ Flanking | cry1F Insert | 3′ Flanking |
| 281-24-236 | 1-2074 | 2,075-12,748 | 12,749-15,490 |

This insertion event, including components thereof, is further illustrated in, for example, U.S. Pat. No. 7,179,965. Seed deposit information is also provided therein. Based on these insert and border sequences, event-specific primers were, and can be, generated. PCR analysis demonstrated that these cotton lines can be identified in different cotton genotypes by analysis of the PCR amplicons generated with these event-specific primer sets. Thus, these and other related procedures can be used to uniquely identify these cotton lines.

As used herein, a “line” is a group of plants that display little or no genetic variation between individuals for at least one trait. Such lines may be created by several generations of self-pollination and selection, or vegetative propagation from a single parent using tissue or cell culture techniques.

As used herein, the terms “cultivar” and “variety” are synonymous and refer to a line which is used for commercial production. “Stability” or “stable” means that with respect to the given component, the component is maintained from generation to generation and, preferably, at least three generations at substantially the same level, e.g., preferably ±15%, more preferably ±10%, most preferably ±5%. The stability may be affected by temperature, location, stress and the time of planting. Comparison of subsequent generations under field conditions should produce the component in a similar manner.

“Commercial Utility” is defined as having good plant vigor and high fertility, such that the crop can be produced by farmers using conventional farming equipment, and the oil with the described components can be extracted from the seed using conventional crushing and extraction equipment. To be commercially useful, the yield, as measured by seed weight, oil content, and total oil produced per acre, is within 15% of the average yield of an otherwise comparable commercial canola variety without the premium value traits grown in the same region.

“Agronomically elite” means that a line has desirable agronomic characteristics such as yield, maturity, disease resistance, and the like, in addition to the insect resistance due to the subject event(s).

As one skilled in the art will recognize in light of this disclosure, preferred embodiments of detection kits, for example, can include probes and/or primers directed to and/or comprising “junction sequences” or “transition sequences” (where the cotton genomic flanking sequence meets the insert sequence). For example, this includes a polynucleotide probe, primer, or amplicon comprising a sequence including residues, as indicated in Table 1. Some preferred primers can include at least ˜15 residues of the adjacent flanking sequence and at least ˜15 residues of the adjacent insert sequence. Residues within 200 bases or so of the junction sequences can be targeted. With this arrangement, another primer in either the flanking or insert region can be used to generate a detectable amplicon that indicates the presence of an event of the subject invention. In some preferred embodiments, one primer binds in the flanking region and one binds in the insert, and these primers can be used to generate an amplicon that spans (and includes) a junction sequence.

One skilled in the art will also recognize that primers and probes can be designed to hybridize, under a range of standard hybridization and/or PCR conditions, to a segment of SEQ ID NO:1, and complements thereof, wherein the primer or probe is not perfectly complementary to the exemplified sequence. That is, some degree of mismatch can be tolerated. For an approximately 20 nucleotide primer, for example, typically one or two or so nucleotides do not need to bind with the opposite strand if the mismatched base is internal or on the end of the primer that is opposite the amplicon. Various appropriate hybridization conditions are provided below. Synthetic nucleotide analogs, such as inosine, can also be used in probes. Peptide nucleic acid (PNA) probes, as well as DNA and RNA probes, can also be used. What is important is that such probes and primers are diagnostic for (able to uniquely identify and distinguish) the presence of an event of the subject invention.

Components of the transgene “insert” or construct are disclosed in, for example, U.S. Pat. No. 7,179,965. Polynucleotide sequences or fragments of these components can be used as DNA primers or probes in the methods of the present invention.

In some embodiments of the invention, compositions and methods are provided for detecting the number of copies of the transgene/genomic insertion region, in plants and seeds and the like, from a cotton plant designated WIDESTRIKE comprising Cry1F event 281-24-236. DNA sequences are provided that comprise at least one transgene/genomic insertion region junction sequence provided herein in SEQ ID NO:1, segments thereof, and complements of the exemplified sequences and any segments thereof. The insertion region junction sequence spans the junction between heterologous DNA inserted into the genome and the DNA from the cotton cell flanking the insertion site. Such sequences are diagnostic for the subject event.

Based on these insert and border sequences, event-specific primers were generated. TaqMan PCR analysis of the subject invention demonstrated that cotton event 281-24-236 can be identified in different cotton lines and genotypes by analysis of the PCR amplicons generated with these event-specific primer sets. These and other related procedures can be used to uniquely identify these cotton lines.

In some embodiments, DNA sequences that comprise (or are complementary, at least in part) to a contiguous portion/segment of the transgene/genomic insertion regions are an aspect of this invention. Included are DNA sequences that comprise a sufficient length of polynucleotides of transgene insert sequence and a sufficient length of polynucleotides of cotton genomic sequence from one or more of the subject cotton plants.

Related embodiments pertain to DNA sequences that comprise at least 2, 3, 4, 5, 6, 7, 8, 9, 10, 11, 12, 13, 14, 15, 16, 17, 18, 19, 20, 21, 22, 23, 24, 25, or more contiguous nucleotides of a transgene portion of a DNA sequence of SEQ ID NO:1, or complements thereof, and a similar length of flanking cotton DNA sequence, or complements thereof. Such sequences are useful as, for example, DNA primers in DNA amplification methods. Components of the invention also include the amplicons produced by such DNA primers and homologous primers.

This invention also includes methods of detecting the presence of DNA, in a sample, from at least one of the cotton plants referred to herein. Such methods can comprise: (a) contacting the sample comprising DNA with a primer set that, when used in a nucleic acid amplification reaction, of the subject invention, with DNA from at least one of these cotton events; (b) performing a TAQMAN PCR amplification reaction using a reference gene identified herein; and (c) analyzing the results.

In still further embodiments, the subject invention includes methods of producing a cotton plant comprising a cry1F event of the subject invention, wherein said method comprises the steps of: (a) sexually crossing a first parental cotton line (comprising an expression cassettes of the present invention, which confers said insect resistance trait to plants of said line) and a second parental cotton line (that lacks this insect tolerance trait) thereby producing a plurality of progeny plants; and (b) selecting a progeny plant based on results of at least one assay technique of the subject invention. Such methods may optionally comprise the further step of back-crossing the progeny plant to the second parental cotton line to producing a true-breeding cotton plant that comprises said insect tolerance trait. According to another aspect of the invention, related methods of determining the zygosity of progeny of a cross are provided.

DNA detection kits can be developed using the compositions disclosed herein and methods well known in the art of DNA detection. The kits are useful for identification of the subject cotton event DNA in a sample and can be applied to methods for breeding cotton plants containing this DNA. The kits contain DNA sequences homologous or complementary to the amplicons, for example, disclosed herein, or to DNA sequences homologous or complementary to DNA contained in the transgene genetic elements of the subject events. These DNA sequences can be used in DNA amplification reactions or as probes in a DNA hybridization method. The kits may also contain the reagents and materials necessary for the performance of the detection method.

A “probe” is an isolated nucleic acid molecule to which is attached a conventional detectable label or reporter molecule (such as a radioactive isotope, ligand, chemiluminescent agent, or enzyme). Such a probe is complementary to a strand of a target nucleic acid, in the case of the present invention, to a strand of genomic DNA from one of said cotton events, whether from a cotton plant or from a sample that includes DNA from the event. Probes according to the present invention include not only deoxyribonucleic or ribonucleic acids but also polyamides and other probe materials that bind specifically to a target DNA sequence and can be used to detect the presence of that target DNA sequence.

“Primers” are isolated nucleic acids that are annealed to a complementary target DNA strand by nucleic acid hybridization to form a hybrid between the primer and the target DNA strand, and can be used in conjunction with a polymerase, e.g., a DNA polymerase. Primer pairs of the present invention refer to their use for amplification of a target nucleic acid sequence, e.g., by the polymerase chain reaction (PCR) or other conventional nucleic-acid amplification methods.

Probes and primers (and amplicons) are generally 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 polynucleotides or more in length. Such probes and primers hybridize specifically to a target sequence under high stringency hybridization conditions. Preferably, probes and primers according to the present invention have complete sequence similarity with the target sequence, although probes differing from the target sequence and that retain the ability to hybridize to target sequences may be designed by conventional methods.

Methods for preparing and using probes and primers are described, for example, in Molecular Cloning: A Laboratory Manual, 2nd ed., vol. 1-3, ed. Sambrook et al., Cold Spring Harbor Laboratory Press, Cold Spring Harbor, N.Y., 1989. PCR-primer pairs can be derived from a known sequence, for example, by using computer programs intended for that purpose.

Primers and probes based on the flanking DNA and insert sequences disclosed herein can be used to confirm (and, if necessary, to correct) the disclosed sequences by conventional methods, e.g., by re-cloning and sequencing such sequences.

The nucleic acid probes and primers of the present invention hybridize under stringent conditions to a target DNA sequence. Any conventional nucleic acid hybridization or amplification method can be used to identify the presence of DNA from a transgenic event in a sample. Nucleic acid molecules or fragments thereof are capable of specifically hybridizing to other nucleic acid molecules under certain circumstances. As used herein, two nucleic acid molecules are said to be capable of specifically hybridizing to one another if the two molecules are capable of forming an anti-parallel, double-stranded nucleic acid structure. A nucleic acid molecule is said to be the “complement” of another nucleic acid molecule if they exhibit complete complementarity. As used herein, molecules are said to exhibit “complete complementarity” when every nucleotide of one of the molecules is complementary to a nucleotide of the other. Two molecules are said to be “minimally complementary” if they can hybridize to one another with sufficient stability to permit them to remain annealed to one another under at least conventional “low-stringency” conditions. Similarly, the molecules are said to be “complementary” if they can hybridize to one another with sufficient stability to permit them to remain annealed to one another under conventional “high-stringency” conditions. Conventional stringency conditions are described by Sambrook et al., 1989. Departures from complete complementarity are therefore permissible, as long as such departures do not completely preclude the capacity of the molecules to form a double-stranded structure. In order for a nucleic acid molecule to serve as a primer or probe it need only be sufficiently complementary in sequence to be able to form a stable double-stranded structure under the particular solvent and salt concentrations employed.

As used herein, a substantially homologous sequence is a nucleic acid sequence that will specifically hybridize to the complement of the nucleic acid sequence to which it is being compared under high stringency conditions. The term “stringent conditions” is functionally defined with regard to the hybridization of a nucleic-acid probe to a target nucleic acid (i.e., to a particular nucleic-acid sequence of interest) by the specific hybridization procedure discussed in Sambrook et al., 1989, at 9.52-9.55. See also, Sambrook et al., 1989 at 9.47-9.52 and 9.56-9.58. Accordingly, the nucleotide sequences of the invention may be used for their ability to selectively form duplex molecules with complementary stretches of DNA fragments.

Depending on the application envisioned, one can use varying conditions of hybridization to achieve varying degrees of selectivity of probe towards target sequence. For applications requiring high selectivity, one will typically employ relatively stringent conditions to form the hybrids, e.g., one will select relatively low salt and/or high temperature conditions, such as provided by about 0.02 M to about 0.15 M NaCl at temperatures of about 50° C. to about 70° C. Stringent conditions, for example, could involve washing the hybridization filter at least twice with high-stringency wash buffer (0.2×SSC, 0.1% SDS, 65° C.). Appropriate stringency conditions which promote DNA hybridization, for example, 6.0× sodium chloride/sodium citrate (SSC) at about 45° C., followed by a wash of 2.0×SSC at 50° C. are known to those skilled in the art, 6.3.1-6.3.6. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or either the temperature or the salt concentration may be held constant while the other variable is changed. Such selective conditions tolerate little, if any, mismatch between the probe and the template or target strand. Detection of DNA sequences via hybridization is well-known to those of skill in the art, and the teachings of U.S. Pat. Nos. 4,965,188 and 5,176,995 are exemplary of the methods of hybridization analyses.

In a particularly preferred embodiment, a nucleic acid of the present invention will specifically hybridize to one or more of the primers (or amplicons or other sequences) exemplified or suggested herein, including complements and fragments thereof, under high stringency conditions. In one aspect of the present invention, a nucleic acid molecule of the present invention has the nucleic acid sequence set forth in SEQ ID NOs:2-6, or complements and/or fragments thereof.

In another aspect of the present invention, a marker nucleic acid molecule of the present invention shares between 80% and 100% or 90% and 100% sequence identity with such nucleic acid sequences. In a further aspect of the present invention, a nucleic acid molecule of the present invention shares between 95% and 100% sequence identity with such sequence. Such sequences may be used in plant breeding methods, for example, to identify the progeny of genetic crosses. The hybridization of the probe to the target DNA molecule can be detected by any number of methods known to those skilled in the art, these can include, but are not limited to, fluorescent tags, radioactive tags, antibody based tags, and chemiluminescent tags.

Regarding the amplification of a target nucleic acid sequence (e.g., by PCR) using a particular amplification primer pair, “stringent conditions” are conditions that permit the primer pair to hybridize only to the target nucleic-acid sequence to which a primer having the corresponding wild-type sequence (or its complement) would bind and preferably to produce a unique amplification product, the amplicon.

The term “specific for (a target sequence)” indicates that a probe or primer hybridizes under stringent hybridization conditions only to the target sequence in a sample comprising the target sequence.

As used herein, “amplified DNA” or “amplicon” refers to the product of nucleic-acid amplification of a target nucleic acid sequence that is part of a nucleic acid template. For example, to determine whether the cotton plant resulting from a sexual cross contains transgenic event genomic DNA from the cotton plant of the present invention, DNA extracted from a cotton plant tissue sample may be subjected to nucleic acid amplification method using a primer pair that includes a primer derived from flanking sequence in the genome of the plant adjacent to the insertion site of inserted heterologous DNA, and a second primer derived from the inserted heterologous DNA to produce an amplicon that is diagnostic for the presence of the event DNA. The amplicon is of a length and has a sequence that is also diagnostic for the event. The amplicon may range in length from the combined length of the primer pairs plus one nucleotide base pair, and/or the combined length of the primer pairs plus about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500, 750, 1000, 1250, 1500, 1750, 2000, or more nucleotide base pairs (plus or minus any of the increments listed above). Alternatively, a primer pair can be derived from flanking sequence on both sides of the inserted DNA so as to produce an amplicon that includes the entire insert nucleotide sequence. A member of a primer pair derived from the plant genomic sequence may be located a distance from the inserted DNA sequence. This distance can range from one nucleotide base pair up to about twenty thousand nucleotide base pairs. The use of the term “amplicon” specifically excludes primer dimers that may be formed in the DNA thermal amplification reaction.

Nucleic-acid amplification can be accomplished by any of the various nucleic-acid amplification methods known in the art, including the polymerase chain reaction (PCR). A variety of amplification methods are known in the art and are described, inter alia, in U.S. Pat. No. 4,683,195 and U.S. Pat. No. 4,683,202. PCR amplification methods have been developed to amplify up to 22 kb of genomic DNA. These methods as well as other methods known in the art of DNA amplification may be used in the practice of the present invention. The sequence of the heterologous transgene DNA insert or flanking genomic sequence from a subject cotton event can be verified (and corrected if necessary) by amplifying such sequences from the event using primers derived from the sequences provided herein followed by standard DNA sequencing of the PCR amplicon or of the cloned DNA.

The amplicon produced by these methods may be detected by a plurality of techniques. Agarose gel electrophoresis and staining with ethidium bromide is a common well known method of detecting DNA amplicons. Another such method is Genetic Bit Analysis where a DNA oligonucleotide is designed which overlaps both the adjacent flanking genomic DNA sequence and the inserted DNA sequence. The oligonucleotide is immobilized in wells of a microwell plate. Following PCR of the region of interest (using one primer in the inserted sequence and one in the adjacent flanking genomic sequence), a single-stranded PCR product can be hybridized to the immobilized oligonucleotide and serve as a template for a single base extension reaction using a DNA polymerase and labeled ddNTPs specific for the expected next base. Readout may be fluorescent or ELISA-based. A signal indicates presence of the insert/flanking sequence due to successful amplification, hybridization, and single base extension.

All patents, patent applications, provisional applications, and publications referred to or cited herein are incorporated by reference in their entirety to the extent they are not inconsistent with the explicit teachings of this specification.

### EXAMPLE

Leaf samples of WIDESTRIKE Event 281-24-236 were harvested. Fully expanded leaves of the cotton plant were selected. Four discs were punched from the leaf using a hand held paper puncher. Genomic DNA was isolated from the leaf tissue using the DNeasy 96-well kit (Qiagen, Valencia, Calif.) via a modified protocol. The protocol was modified by the addition of 10 mM sodium metabisulfite (Na₂S₂O₅) to the lysis buffer to prevent the oxidation of polyphenolics. Isolated genomic DNA was quantified using the Quant-iT PicoGreen quantification kit.

Multiple primer and probe combinations were designed and tested. The combination resulting in the most robust signal capable of discriminating between wild-type allele and transgene were selected.

A Master Mix was prepared to run the biplex reaction for detection of Event 281-24-236. The following reagents were included in the cocktail: 12.5 μl of Taqman Genotyping Master Mix; 0.45 μl of WT_F1 primer, 100 μM; 0.225 μl of WT_R2 primer, 100 μM; 0.225 μl of 281_R primer, 100 μM; 0.05 μl of Wt_{—Probe,} 100 μM; 0.05 μl of 281_Probe, 100 μM; 3 μl of DNA; and, 8.5 μl of water. Table 2 lists the following samples of DNA that were used in independent reactions and the purpose for the incorporation of these samples into the experiment. Table 3 lists the sequences of the primers and probes used in the assay. The reactions were amplified in a GenAmp PCR System 9700 using the following conditions: 15 minutes at 95° C. for 1 cycle; 15 seconds at 92° C. then 60 seconds at 60° C. for 31 cycles. Results were measured using a fluorometer. The FAM dye was measured with an excitation of 485 nm and an emission of 535 nm. The VIC dye was measured with an excitation of 525 nm and an emission of 560 nm.

TABLE 2
Positive and Negative Controls.

| Type of Control | Description | Expected Result | Interpretation |
|---|---|---|---|
| Master mix | No DNA is added to | Background RFU | Mix is not |
| negative control | the reaction. | readings. No PCR | contaminated. |
| | | products | |
| Homozygous | Genomic DNA | RFU readings of FAM | Control shows |
| DNA positive | sample known to be | are at least 1 unit higher | amplification of the |
| control (Event | homozygous for the | than that of the non- | trangene (FAM) and |
| 281-24-236) | transgenic sequence | transgenic control. | the reference (VIC) |
| | is added. | Readings of VIC less | alleles from genomic |
| | | than non-transgenic | DNA. |
| | | control. | |
| Hemizygous | Genomic DNA | RFU readings of FAM | Control shows |
| DNA positive | sample known to be | are at least 0.5 unit higher | amplification of the |
| control (Event | hemizygous for the | than that of the non- | transgene (FAM) and |
| 281-24-236) | transgenic and wild- | transgenic control. | the reference (VIC) |
| | type sequences are | Readings of VIC similar | alleles from genomic |
| | added. | to non-transgenic control. | DNA. |
| Non-transgenic | Genomic DNA | Fluorescence readings of | Control only shows |
| DNA negative | sample extracted | VIC are at least 0.5-1 unit | amplification of the |
| control | from a non- | higher than that of the | reference (VIC) |
| | transgenic line of the | negative background | alleles and not the |
| | same background as | control. | transgene (FAM) |
| | unknowns is added. | | from genomic DNA. |

TABLE 3
Primer and Probe Sequences.

| Primer or | |
|---|---|
| Probe Name | Sequence |
| 281_probe | 6FAM-CCCGGACATGAAGCCAT-MGBNFQ |
| (SEQ ID NO: 2) | |
| WT_probe | VIC-ACCCACCATGAACGAGCA-MGBNFQ |
| (SEQ ID NO: 3) | |
| WT_F1 | GCTAAATTGTAAAAGAAAGTTAAAATAAGAGACTCG |
| (SEQ ID NO: 4) | AA |
| WT_R2 | GGTTAATAAAGTCAGATTAGAGGGAGACAA |
| (SEQ ID NO: 5) | |
| 281_R | CCATCATACTCATTGCTGATCCATGT |
| (SEQ ID NO: 6) | |

Amplicons generated from these primers are as follows. For the 281 amplicon (SEQ ID NO:7), the underlined base indicates the start of the transgene insert.

(SEQ ID NO: 7)
[GCTAAATTGTAAAAGAAAGTTAAAATAAGAGACTCGAA>TTATAATAT
GATTCTCTGGCGCACTAATTAAGCTACTATATATTGTCAATAGTATTGT
AA<ATGGCTTCATGTCCGGG]AAATCT<ACATGGATCAGCAATGAGTAT
GATGG].

Brackets indicate the direction and location of SEQ ID NO:4, SEQ ID NO:2, and SEQ ID NO:6, respectively.

For the wild-type amplicon (SEQ ID NO:8), the underlined bases indicate the deletion when the transgene inserts.

(SEQ ID NO: 8)
[GCTAAATTGTAAAAGAAAGTTAAAATAAGAGACTCGAA>TTATAATAT
GATTCTCTGRCGCACTAATTAAGCTACTATATATTGTCAATAGTATNNN
AACTTCTGTAGCTCTATTCCTTAG[ACCCACCATGAACGAGCA>GATAA
ATGCTTNNNNNAC<TTGTCTCCCTCTAATCTGACTTTATTAACC].

Brackets indicate the direction and location of SEQ ID NO:4, SEQ ID NO:3, and SEQ ID NO:5, respectively.

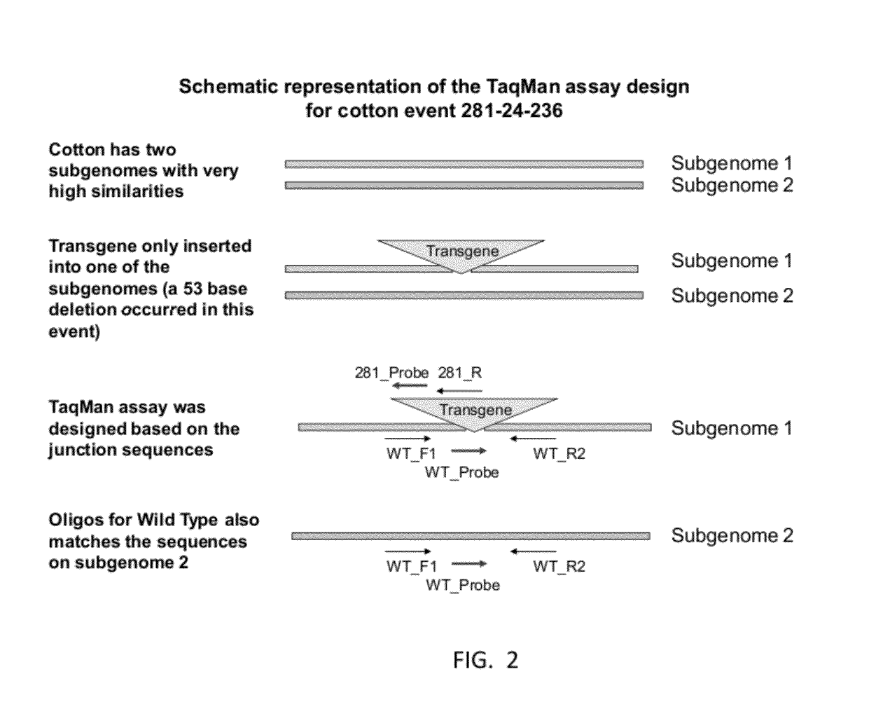

FIG. 2 illustrates location of the primer and probe sequences with respect to the subgenomes and trangene insert.

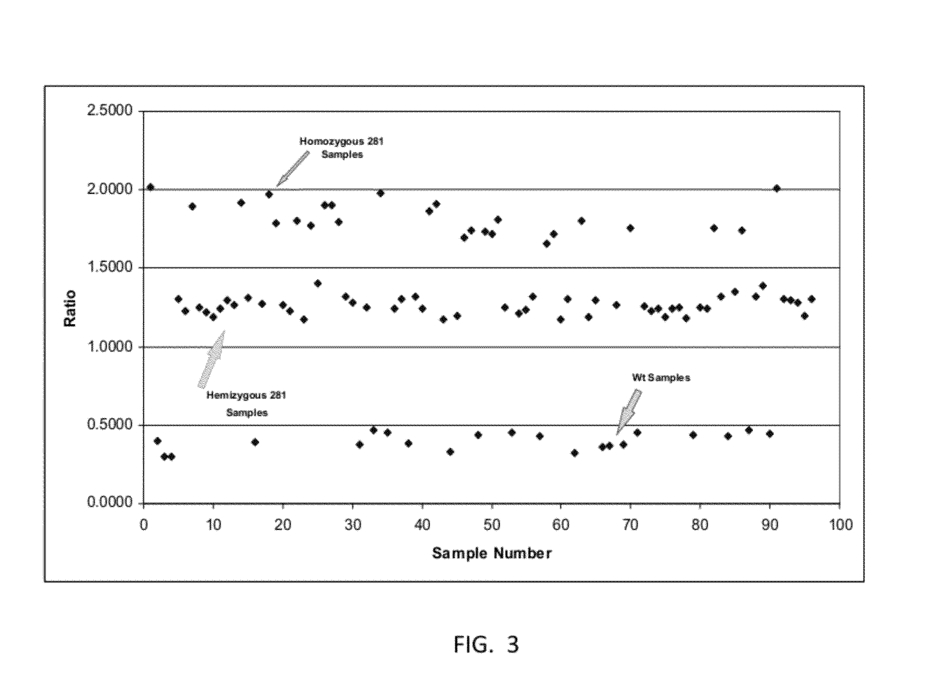

Following completion of the TaqMan PCR and fluorescence reading, a table and distribution graph were generated. The wild-type, hemizygous, and homozygous controls of similar genotypic background served as negative and positive controls. In a segregating population, three clusters of data points should be obtained allowing the cut-off points to be visually determined. These cut-off points are arbitrary, and separation between clusters is usually about 0.5-1.0 unit. However, data points could scatter due to variability in the assay. For this experiment, three clusters of data points were clearly visible (FIG. 3).

FIG. 3 shows cry1F zygosity determination with the endpoint TaqMan assay. On completion of PCR and fluorescence readings, a distribution graph was generated as follows: SOB1=Signal Over Background of FAM (sample signal over average of background signal at 535 nm), and SOB2=Signal Over Background of VIC (sample signal over average of background signal at 560 nm). Genotype calls were made with SOB1/SOB2<0.5 for wild type, 0.5<SOB1/SOB2<1.5 for hemizygotes, and SOB1/SOB2>1.5 for homozygotes.

The wild type control samples that do not contain Event 281-24-236 genomic DNA resulted in relative fluorescent unit (RFU) readings from amplification of the reference gene. Samples containing hemizygous or homozygous Event 281-24-236 genomic DNA resulted in RFU readings for the FAM probe at least 0.5-1 unit higher than that of the negative background control. The data points for wild-type are less than 0.5, those for hemizygous samples range from 0.5-1.5, and those for homozygous are above 1.5.

This protocol can be used to detect Event 281-24-236 cotton plants which contain the cry1F transgene. In one assay oligonucleotides specific to Event 281-24-236 and its flanking genomic sequences are used to amplify a 147 bp fragment, and in the second assay oligonucleotides corresponding to cotton genomic DNA are used as a reference sequence to amplify a 186 bp fragment in a single bi-plex reaction. Zygosity is determined by the relative intensity of fluorescence specific for Event 281-24-236 to the reference sequence. A summary of the above Example appears in Table 4.

TABLE 4
Probes used to determine zygosity of Event 281-24-236.

| | | Homozygous 281 | | |
|---|---|---|---|---|
| Oligos | purpose | samples | hemizygous 281 | Wt samples |
| WT_F1 + 281_probe + 281_R | detecting transgene | positive from | positive from | Negative |
| | event 281-24-236 | subgenome 1 with two | subgenome 1 with one | |
| | | copies | copy | |
| WT_F1 + WT_probe + WT_R2 | detecting wild type | positive from | positive from | positive from both |
| | allele | subgenome 2 with two | subgenome 1 with one | subgenomes with |
| | | copies | copy and subgenome 2 | four copies |
| | | | with two copies | |

1. A method for determining zygosity of a cotton plant comprising a 281-24-236 event, said 281-24-236 event comprising a transgene construct comprising a cry1F gene, said method comprising:
obtaining a sample of genomic DNA from said cotton plant,
contacting said sample with:
a. a first flanking primer that hybridizes between residues 1-2074 of SEQ ID NO:1 or the complement thereof
b. a second flanking primer that hybridizes between residues 12,749-15,490 of SEQ ID NO:1 or the complement thereof
c. a transgene primer that hybridizes between residues 2075-12,748 of SEQ ID NO:1 or the complement thereof
wherein said first flanking primer and said second flanking primer form a wild-type amplicon when subjected to PCR conditions,
wherein said transgene primer forms a transgene amplicon with said first flanking primer or said second flanking primer when subjected to PCR conditions,
further contacting said sample with
d. a florescent event probe that hybridizes with said transgene amplicon
e. a florescent wild-type probe that hybridizes with said wild-type amplicon subjecting said sample to fluorescence-based endpoint TaqMan PCR conditions, quantitating said florescent event probe that hybridized to said event amplicon, quantitating said florescent wild-type probe that hybridized to said wild-type amplicon,
comparing amounts of hybridized florescent event probe to hybridized florescent wild-type probe; and
determining zygosity of said cotton tissue by comparing florescence ratios of hybridized fluorescent event probe and hybridized fluorescent wild-type probe.

2. The method of claim 1, wherein said plant comprises a first subgenome and a second subgenome, said transgene amplicon being formed from said first subgenome, and said wild-type amplicon being formed from said second subgenome.

3. The method of claim 1, wherein said transgene amplicon and said wild-type amplicon are between 100-500 basepairs in length.

4. The method of claim 1, wherein said transgene amplicon and said wild-type amplicon are between 100-200 basepairs in length.

5. The method of claim 1, wherein said transgene amplicon consists of 147 basepairs, and said wild-type amplicon consists of 186 basepairs.

6. The method of claim 1, wherein said primers are 10-50 basepairs in length.

7. The method of claim 1, wherein said probes are 10-30 basepairs in length.

8. The method of claim 5 wherein said method is used for breeding introgression of the 281-24-236 event into another cotton line.

9. The method of claim 8, wherein said another cotton line lacks said 281-24-236 event.

10. The method of claim 1 wherein results of said method are read directly in a plate reader.

11. The method of 1 wherein said sample is obtained from a cotton plant in a field.

12. The method of claim 1 wherein said probes are labeled with a fluorescent dye and quencher.

13. The method of claim 12 wherein said transgene probe comprises FAM as said fluorescent dye at the 5′ end of said transgene probe and said quencher is selected from the group consisting of MGBNFQ and a Black Hole Quencher 1 (BHQ1) as said quencher on the 3′ end of said transgene probe.

14. The method of claim 1 wherein said wild-type probe is labeled with VIC at the 5′ end of said wild-type probe and said quencher is selected from the group consisting of MGBNFQ and a Black Hole Quencher 2 (BHQ2) at the 3′ end of said wild-type probe.

15. The method of claim 1 wherein said wild-type probe comprises SEQ ID NO:3.

16. The method of claim 1 wherein said transgene probe comprises SEQ ID NO:2.

17. The method of claim 1 wherein said first flanking primer comprises SEQ ID NO:4, said second flanking primer comprises SEQ ID NO:5, and said transgene primer comprises SEQ ID NO:6.

18. The method of claim 1 wherein said transgene amplicon comprises SEQ ID NO:7.

19. The method of claim 1 wherein said wild-type amplicon comprises SEQ ID NO:8.

20. A kit for performing the method of claim 1, said kit comprising:
a first flanking primer that hybridizes between residues 1-2074 of SEQ ID NO:1 or the complement thereof;
a second flanking primer that hybridizes between residues 12,749-15,490 of SEQ ID NO:1 or the complement thereof, wherein said first flanking primer and said second flanking primer form a wild-type amplicon when subjected to PCR conditions;
a transgene primer that hybridizes between residues 2075-12,748 of SEQ ID NO:1 or the complement thereof, wherein said transgene primer forms a transgene amplicon with said first flanking primer or said second flanking primer when subjected to PCR conditions;
a florescent event probe that hybridizes with said transgene amplicon; and
a florescent wild-type probe that hybridizes with said wild-type amplicon.


SEQUENCE LISTING (8 sequences)

SEQ ID NO: 1 - DNA, 15490 bp, Artificial Sequence
  Cry1F Event 281-24-236 including the insert and flanking sequences
aagcttgcttaaaagtatcacaagccatgatccttataaaaatgatatctgacactatgcttctttgcacattcttcactatgctttctcatttgagcaatggtgggatttgctctcaaatttggtggccctatgtcagtattcaaaaattttcataggtcaaaggcttgaagataagccttcatttttaccacccatatgtagtaaatttcaccgatgaatacaagaggtggaggtggtgagaaactagatgaatacattcttgcagaaaacgtccctcaaagatcaaaatggctctcaataccaattcttggtgttttcagactaagagagaaagcaaaacgagacaatgaacccacaagatgaattaatttcaatacatttttttaaatttcatttcaaacggttacaatatataccttttgttttccaaacaactcaactgatccaactaacatcttggaaataggaaaacttaacttgtacacaaactgattgtgaaatcaacacctaaacacatcaagtcattaccaacttagtttattcctagctaagtatcttaacataaggattgaacaaaaggttaaaccaaaactttgatttttttgctctaggaagacagcttgcacttcatcatacagttcgccttattgttaatcaactcacactttggctgtccgttgatatgctggcagttaaaatgttcaccaatgtgcttagacaatccgccaagcctatcgtgtaaaaactctttgcatgacagttcacctattgacagtccgccagtcttttaatgctcagactgtctaccatgtgacttagagacggaattgtttgccatatgtgttcgccaactagattgttcaccatttgtgttgtttaccatgtgtgttcgctaatgcatggttggccatgtcgcaaaacaaatttttggatgggccaaaattggaattttttcatttgaccaaatataaaaaaacgaattgaacaaattacttttagagggattaaaaattaaaattatactatttatcgaggggagtcaaggctcctatcttcttcgcttcttctattgtttagattaagactaaaattttaaaatttatagaaattaaaattgatgaaattaaaatacaaaattaaatatataattcagttaggtttaaccattttttaatgttgcttagctttaatgtttgggatttggctactttcagtcgttatgcagttatgctcagacaaatttgttctctttctgtcttatcaactactcaaaatctcagtatagttatgtcatttaatctcttcatcgtagatgttatattggtgaaaatggggccaagaaatccacattcaatgactttgaaagaatatatattgttagttgcacattcccttattcaatcacagttgcttgtttctgagtctatagaatcatgatatttgtaaatcttatataaagtaagagtatatggctagacagtctggccctgtcggctgaaggtagggaggaattaatcaatcacagttgcttgtttctgagtctatagaatcatgaattttaaatttatggaatgcattttttcgaagatattgtatgcattaagtgtaattttagtttcaatatgaaatttgagatttatatatatacttacataaaaccctcctttactgaattagtgccatggataaaagaccaattaagcaatccttccaacacgtgcatgcactggattttcatcgcctcgtccattgttaaattgataggttaataagaacaattagttggctactgattatatggattctgggttaaaagtatttaggtttactgttacatacatggaggatctacatctattttcacttttgtttaattaatttaagttagttttgatgagtttaaggattgtactagccaatagtagtacataaaggagatagagtaccaaaacaaagaaaaagccgaaaggtgttaatgctaaattgtaaaagaaagttaaaataagagactcgaattataatatgattctctggcgcactaattaagctactatatattgtcaatagtattgtaaatggcttcatgtccgggaaatctacatggatcagcaatgagtatgatggtcaatatggagaaaaagaaagagtaattaccaattttttttcaattcaaaaatgtagatgtccgcagcgttattataaaatgaaagtacattttgataaaacgacaaattacgatccgtcgtatttataggcgaaagcaataaacaaattattctaattcggaaatctttatttcgacgtgtctacattcacgtccaaatgggggccacttggctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctccttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgacatgtctccggagaggagaccagttgagattaggccagctacagcagctgatatggccgcggtttgtgatatcgttaaccattacattgagacgtctacagtgaactttaggacagagccacaaacaccacaagagtggattgatgatctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggatccacattgtacacacatttgcttaagtctatggaggcgcaaggttttaagtctgtggttgctgttataggccttccaaacgatccatctgttaggttgcatgaggctttgggatacacagcccggggtacattgcgcgcagctggatacaagcatggtggatggcatgatgttggtttttggcaaagggattttgagttgccagctcctccaaggccagttaggccagttacccagatctgagtcgacggatccccgacatatgccccggtttcgttgcgactaacatgagttcttggacaaatttgattggacctgatgagatgatccaacccgaggatatagcaaagctcgttcgtgcagcaatggaacggccaaaccgtgcttttgtccccaagaatgaggtgctatgcatgaaggaatctacccgttgatgtccaacagtctcagggttaatgtctatgtatcttaaataatgttgtcggtattttgtaatctcatatagattttcactgtgcgacgcaaaaatattaaataaatattattattatctacgttttgattgagatatcatcaatattataataaaaatatccattaaacacgatttgatacaaatgacagtcaataatctgatttgaatatttattaattgtaacgaattacataaagatcgaatagaaaatactgcactgcaaatgaaaattaacacatactaataaatgcgtcaaatatctttgccaagatcaagcggagtgagggcctcatatccggtctcagttacaagcacggtatccccgaagcgcgctccaccaatgccctcgacatagatgccgggctcgacgctgaggacattgcctaccttgagcatggtctcagcgccggctttaagctcaatcccatcccaatctgaatatcctatcccgcgcccagtccggtgtaagaacgggtctgtccatccacctctgttgggaattctgatcttggcggtacccggggatcctcattcctccatcagaagtaactccacgctatcaacaatgaatgttccttccgtttctccaatctcaatccaaaccttgtcggtttctggaaagtactctaactctttggtgacatagccggctggtaacggtgtgtagtccccatagcctctgttagattcgcaaggattgtccctacgtccatcggtgtaagccttctcctcataggctgatgcatagtcagcgggtacagaagagttgctctcataggctccatcgtatcctcgattgcgagaagtgtaagtaccctcatactcctcttgagtcgcagtgtagtcattgcaagttacggtgttgtttgggtagacttcctcctcgacgcagttgctgaacttcagctcgtcggtgttgttctcaatctcgtgtatggtgacgcaaccttctccgtatccttctttgtacgcggtaacacgaagaatgtagccacgaccaggacagacacgaacttcttgtgaaacttctgcttcccactcaggaacaacaaggacagagcggtgattgttctgttcttctacatctacgtgccctttcacattccagcaggataggccattgttgaagtcaccattcttgatgacattcctcgcatcatacaaggagaatgcagtgaagatgcgcccttctaactcttcaaagatagcagcattgacacccggaatcacgctaagttcaggaaggtaagcttcccgaatgctatgaacgcgtttgtctgcagcatgaatcatagctatgttggtatcagcttggagcctatcatactgagagttcacaaacagagcgtcaacgctttctttggcttctttgtacacaatgtttgtttcccattccaacttctctctcttgtccctccacttcttctcagccctcttcactctagcgagggcttctccaacaagtggtttctcttctagaaactccagattgcctagcctggcatggccatcttgagtcttgatcttgaagatcacccacacaccgaggtcttcgttcaggtcggtacagccaacgtctatgtccaaggagaagtggtgtgagtgatgggcacacttgccgatgggacttggggctgaaagtggccagagtgaacccgtcccaggcacattgactgtctcatgtttggcgttgtatctgatgaggtagatctcaaggtcttgactgtcctcgatgtaacctctcaactggtatcttgtgtaggctttgagtttcgattcatctatcttctggtacaggtatgttggatagcactcatcaaaggtacccaagagcgtaacatagttctccttgaacacatcatcacctccttgaatggtgatgtccgtacttcccctccatccacgatctagttgcctgttgatcccgcgaaagttgggatcttgaagcaagttccgctcatcactaagtcgcttagcatgtttgaccttctcggacaactccttcttctcatccaaacagaactcatcagagaggcactcaacaaggttggaaacgcgatcgatgtgatagtcagtcacatctgtcttgagcccaatctgattggacgaagtgaacagagcattcaccgccttctgtgctctttccaagtcagactctgcctcgagtgtggcagtaactggaatcaactcaaacctgtcaatgtacacttcgttgcctgaactgaaggtatcagcacctactgtgaaactgctctggctcattggaaaggtgaacgcggtgttgatagtggcgtaggagaaagattggaatgtaagtggatcaccggtatccattgtcttgttgaactgaccagcaaagatccgttcacctgcaaccgtaacgtagattcttagattggtagtagaggcatagcgtatcctggcacgatacctttggggaagttgcccattgatgttgacaatggtgtacgcgaatggtcctccactagtgcgtcgaagaatgtctcctcccgtgaaccccggccctcttacaactgtagttcctgactgaagtgtgtgtgccttcaccaagggaatctgagtgattctctctggatcaatggtgtttgtgggggtagcgctacgatgcgtccaagagaacattggtgctctccatgagtcggaacctgagatctcacctggccagcgcacaaaggtaacatgattcagcacatgggagtagtcattccaaggtgcgccgctgttgtcttgaggtggtatctcatctagagagtcaatggtcccggagttcctgaatgtgcgggtgtgattcgtaccagtttgttgaaaggccactcccctaagaccgagtacatagtgaggattgccaaagcctcctcggacgaagacaggatctgacaaggtccgatagaaaggacgtggatcttcatctgcaatccagatggcgcccccgggattgaagaccccgtaactaggaaagttgatacgattgccagccgtgttgcgtgagctaactaagtgtcctccccacacagtttgggatctaacagtctctgcagtcacaaacaaagagttcatgaagtccatgagatggggtggtctgactccaaactcagccctgttgaaaccattgggtatgttcgcagaaactggagagtcttcaatgactgaactggtgtagatctcccttgtaagttgggatgacgtttgaatcggataggtacgaacatcgtagttcggaaagagagcaactatgtctaacacagtaagtgtaaggtctctcctgaactgattgaacctggcccattggcgagtgttagtacctctcaggttctccaatccctgattgtaggtatccaaacaatgtttcgtgtatcgatgaatcagattgatgagtctgttgtagtgattgttgacagtagctatgtccagtccccaaccttgcccaaacgacacagcgtcgcgcagtagtgacaagtgcaggttagcagcttgaacatagaccgagagaagagggatctcgaagctggtaagggtgaagttgttgatggctgtgatcaaagcatcatctgtgttagcaaagcgtatacgcacatcttctctcagttgggcattgttaggattggcttcccactctcttagtgcttcaatgtagatctcatagctgtctgctaagccacgaagggtagtgatggcccgattcctttccaaggtctcaatcctttgttcaatcaactgttcaatctggagaagaaagaggctccaatcagatggagtgatgaagccccagatgaggtcgaagaggccaaacgcaactcccacacctggaacaaactcagacaacaggagacgtgtaagggacagggagatgtctaacggcaatctgccagtcgacctctcttcgttgagaatctctacttcaggattgttgaggcagttgtaggggacgcactgattctgtatgttgttctccattgttggatccgcgatttggtgtatcgagattggttatgaaattcagatgctagtgtaatgtattggtaatttgggaagatataataggaagcaaggctatttatccatttctgaaaaggcgaaatggcgtcaccgcgagcgtcacgctctagtcgaccatgtacgtaagcgcttacgtttttggtggaccccctcgaccatgtacgtaagcgcttacgtttttggtggaccccctcgaccatgtacgtaagcgcttacgtttttggtggaccccctcgaccatgtacgtaagcgcttacgtttttggtggaccccctcgacggatcccccctcgaccctagacgtatctattcaaaagtcgttaatggctgcggatcaagaaaaagttggaatagaaacagaatacccgcgaaattcaggcccggttgccatgtcctacacgccgaaataaacgaccaaattagtagaaaaataaaaactgactcggatacttacgtcacgtcttgcgcactgatttgaaaaatctcaatataaacaaagacggccacaagaaaaaaccaaaacaccgatattcattaatcttatctagtttctcaaaaaaattcatatcttccacaccctcgagatctagatatcgatgaattttggcgcgccttaattaaggaattcctcgagtttaaacggatccctgaaagcgacgttggatgttaacatctgcaaattgccttttcttatcgaccatgtacgtaagcgcttacgtttttggtggacccttgaggaaactggtagctgttgtgggcctgtggtctcaagatggatcattaatttccaccttcacctacgatggggggcatcgcaccggtgagtaatattgtacggctaagagcgaatttggcctgtagacctcaattgcgagctttctaatttcaaactattcgagctttctaattgaatatatccagggcccagcgtaagcaataccagccacaacaccctcaacctcagcaaccaaccaagggtatctatcttgcaacctctctagatcatcaatccactcttgtggtgtttgtggctctgtcctaaagttcactgtagacgtctcaatgtaatggttaacgatatcacaaaccgcggccatatcagctgctgtagctggcctaatctcaactggtctcctctccggagacatgtcgacctgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcaaaataattataaaacatacttgtttattataatagataggtactcaaggttagagcatatgaatagatgctgcatatgccatcatgtatatgcatcagtaaaacccacatcaacatgtatacctatcctagatcgatatttccatccatcttaaactcgtaactatgaagatgtatgacacacacatacagttccaaaattaataaatacaccaggtagtttgaaacagtattctactccgatctagaacgaatgaacgaccgcccaaccacaccacatcatcacaaccaagcgaacaaaaagcatctctgtatatgcatcagtaaaacccgcatcaacatgtatacctatcctagatcgatatttccatccatcatcttcaattcgtaactatgaatatgtatggcacacacatacagatccaaaattaataaatccaccaggtagtttgaaacagaattctactccgatctagaacgaccgcccaaccagaccacatcatcacaaccaagacaaaaaaaagcatgaaaagatgacccgacaaacaagtgcacggcatatattgaaataaaggaaaagggcaaaccaaaccctatgcaacgaaacaaaaaaaatcatgaaatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatggaccggaacgccgatctagagaaggtagagaggggggggggggggggaggacgagcggctgtaccttgaagcggaggtgccgacgggtggatttgggggagatctggttgtgtgtgtgtgcgctccgaacaacacgaggttggggaaagagggtgtggagggggtgtctatttattacggcgggcgaggaagggaaagcgaaggagcggtgggaaaggaatcccccgtagctgccggtgccgtgagaggaggaggaggccgcctgccgtgccggctcacgtctgccgctccgccacgcaatttctggatgccgacagcggagcaagtccaacggtggagcggaactctcgagaggggtccagaggcagcgacagagatgccgtgccgtctgcttcgcttggcccgacgcgacgctgctggttcgctggttggtgtccgttagactcgtcgacggcgtttaacaggctggcattatctactcgaaacaagaaaaatgtttccttagtttttttaatttcttaaagggtatttgcttaatttttagtcactttattttattctattttatatctaaattattaaataaaaaaactaaaatagagttttagttttcttaatttagaggctaaaatagaataaaatagatgtactaaaaaaattagtctataaaaaccattaaccctaaaccctaaatggatgtactaataaaatggatgaagtattatataggtgaagctatttgcaaaaaaaaaggagaacacatgcacactaaaaagataaaactgtagagtcctgttgtcaaaatactcaattgtcctttagaccatgtctaactgttcatttatatgattctctaaaacactgatattattgtagtactatagattatattattcgtagagtaaagtttaaatatatgtataaagatagataaactgcacttcaaacaagtgtgacaaaaaaaatatgtggtaattttttataacttagagatgcaatgctcattatctctagagaggggcacgaccgggtcacgctgcactgcagccaagtggcccccatttggacgtgaatgtagacacgtcgaaataaagatttccgaattagaataatttgcttattgctttcgcctataaatacgacggatcgtaatttgtcgctttatcagaatgtactttcattttataataacgctgcggacatctacatttttgaattgaaaaaaaattggtaattactctttctttttctccatattgaccatcatactcattgctgatccatgtagatttcccttacttgtctccctctaatctgactttattaacccaaagcaattgcttatttgttccccacgcccacaaagcccagcattgtccctaaggtattaatttgttgttcgattcttgttcttgaacccatttggagaatgcaacaagggttttcatgtcagcacggtaatggttctgtgtaaattccagtagtgctgcccaagtaaagtctgggtatttcctcgaatttgcggcattaactaagctagctgctggtgtcaccggtctctccagctcgggacatagaaagaatgcaagtgattttctcaccgtttcagtattcactactgcccaagcagctcttgtagataccgtttgtcaaagcctgtattcaaacaccacaacctcattttcgttaaaattttttgtatatacgtatgcatatatgttcagaatgtttattaccatgaatgtatcgcaatgttgacaacgaaagctcctgggatatgagcaacggagtgccacttttcatcggcaaacacttgaaggcctccaacttgatcctgatgaaggattgtcaaggaagtgggatcggtgtggggaccggttcccagggtcaactcaggcttttcgcatggagagtagtgattcagtctcaggattgaatcattttgttcgaaaaagtctttgaagtaggcttggtcaagccctagacttatccctagcagccccattatctctttggaaaccttgttcctggcttcacaatattcctggtaaagcctcctgtcaagtacgaaaagcaaaatcgagtgttagattcctatcctatggtaaaacttgtcgcaacattctacaaattaacataggttataaaagagaaacgcaaagtcaaaaaaggccatccagttataatgtgtattttttgttttaggagaaggggtatttaagcaatccatgttgaatggacttagggttcggtcaatgcaaggaatgatgtctattgaatttgtcaccccatctctttcaaacctaaaaaagatgtatatccaattgtcactatttgtacaaggcttgaaatacacatggggttaacaaatacgctgaaactgcaacatgtattttttgcattttggagactaaatcttgacgtaaaaataagctgatcatctcgtatattgactgaatgcaaattagcatttctcctatttaattaggaatggaagggtgagaagaatttatttacccaaaatctctaaaatcttcagtgccgaaaagacagtgtttctttccatggcaacttggaggaaaacctgccaacgaaactgcttgcatacccatagctttctcctacttttctcttagccttttgcttctcagagagttgcaggctgaagaagcggtccatgtactgatgagcttacaaccaaaaaaaacccatgcttcttgcatgcctcattcaccgcccctgccacttttgatacagcaagagagcccccaagcaagaaagctcccaagtcaatggcttgaattacaagttccgggtcatccaggcatggcttatcgtcgtcaggccatatgaactgagagggtatattggattcagatccaaggattgatgcatcgaaagctaaagggcgatgctcattctttgcgacagcagcagttggcggaagcatcggcttctcttgaatgacagaaactattggcagacaatgtaccattttctgtttcttgttttaggtggtcggagaggaaaaagaagacaagatatagaggagcaaaactaagagggtttaaataagagaagtagaaaaccaataatatttggaatctaattgtttgttttgaactgttgcctcatttcccctttaatttgtgttgtgttggcagcttaacgcttcctttgggttcgattgcaatatagtgcgttgaaatttttaccttgattttcaatctcactacaccacagtgacttgcttggttgtttggataatttttaccttgattttcaatctcactacaccacagtgacttgcttggttgtttggataatttttaccttgattttcaatctcactacaccacagtgacttgcctggttgtttggatatcttggtttccagcacagtgagacaagcctgctgcactcgacaaccttatcttcactggtgctaaaagctccagtcagctctagctagggcaataggtatttttggatgacaaaaatacccatactttaatttattattttaccattttatccttagatgttaaactaatttctttcccaatcttatttgttttcagatttgggaataaaacaatagtttctcccttgttcttgctggtttctcccttgttcttgctggtttctcccctcttttttgctttgttcttggttgtgggagcttaggatattctttttcttcaatcagactaacaagttagagatatcttgtgttttttcactttattttctcatgctcaacatttaccctttttctcaattaacaggggaagtctacattaattagtgcactctcagatagtaatctgtatagtgatgcaatgtatatatattctttaaaacagtttttcctcgaagttaaattctttgttaaaagtaaaaggctggatgtttttacctaattggaggtaatgtctttgtgtagattgtttgcaacattggatggttgattaaaaagtgttgttcttccttcaaggtgagatggtttgctgtcactcactattattattgttgttattgttattgcttttccattgaatagcctggttctaaatgatatacttaccttatcccataggcagcaacattttatcttttgatctttggacctatcatttagcatgctttacactctatttagtaatattattactaactataattttaagtcatacacaatgaaattacctaaacatctaaactcaaaaaattataacttaaagctt

SEQ ID NO: 2 - DNA, 17 bp, Artificial Sequence
  Probe 281_probe
cccggacatgaagccat

SEQ ID NO: 3 - DNA, 18 bp, Artificial Sequence
  Probe WT_probe
acccaccatgaacgagca

SEQ ID NO: 4 - DNA, 38 bp, Artificial Sequence
  Primer WT_F1
gctaaattgtaaaagaaagttaaaataagagactcgaa

SEQ ID NO: 5 - DNA, 30 bp, Artificial Sequence
  Primer WT_R2
ggttaataaagtcagattagagggagacaa

SEQ ID NO: 6 - DNA, 26 bp, Artificial Sequence
  Primer 281_R
ccatcatactcattgctgatccatgt

SEQ ID NO: 7 - DNA, 147 bp, Artificial Sequence
  The 281 Amplicon produced using the WT_F1 primer (SEQ ID NO 4) and the 281_R primer (SEQ ID NO 6)
gctaaattgtaaaagaaagttaaaataagagactcgaattataatatgattctctggcgcactaattaagctactatatattgtcaatagtattgtaaatggcttcatgtccgggaaatctacatggatcagcaatgagtatgatgg

SEQ ID NO: 8 - DNA, 186 bp, Artificial Sequence
  The wild type amplicon produced using primer WT_F1 (SEQ ID NO 4) and WT_R2 (SEQ ID NO 5)
gctaaattgtaaaagaaagttaaaataagagactcgaattataatatgattctctgrcgcactaattaagctactatatattgtcaatagtatnnnaacttctgtagctctattccttagacccaccatgaacgagcagataaatgcttnnnnnacttgtctccctctaatctgactttattaacc